(19) United States
(12) Patent Application Publication
(10) Pub. No.: US 2024/0157113 A1
(43) Pub. Date: May 16, 2024

(54) ROTARY PINCH VALVE FOR FLUID CONTROL

(71) Applicant: Otsuka Medical Devices Co., Ltd., Tokyo (JP)

(72) Inventor: Robin BEK, Campbell, CA (US)

(21) Appl. No.: 18/490,499

(22) Filed: Oct. 19, 2023

Related U.S. Application Data
(60) Provisional application No. 63/383,821, filed on Nov. 15, 2022.

(51) Int. Cl. A61M 39/26; A61M 25/10

(52) U.S. Cl. CPC A61M 39/26; A61M 25/10

(57) ABSTRACT

A fluid control device for an ultrasound-based treatment system having at least one motor having a motor shaft extending from an axis of the motor. The at least one camshaft is operatively coupled to the motor shaft in rotational communication with the motor shaft. The at least one actuator in communication with the at least one camshaft. When the motor shaft is operating, the motor shaft causes the at least one camshaft to rotate, thereby moving the at least one actuator between a first position and a second position through a fluid channel of a cartridge manifold. Other embodiments are also described and claimed.

### PRIORITY CLAIM

This application claims priority to U.S. Provisional Patent Application No. 63/383,821, filed Nov. 15, 2022, titled ROTARY PINCH VALVE FOR FLUID CONTROL, which is incorporated herein by reference.

### BACKGROUND

### Field

This application relates generally to rotary pinch valves and in particular, rotary pinch valves for fluid control.

### Background Information

High blood pressure, also known as hypertension, commonly affects adults. Left untreated, hypertension can result in renal disease, arrhythmias, and heart failure. Treatment of hypertension has focused on interventional approaches to inactivate the renal nerves surrounding a renal artery. Intraluminal devices, such as catheters, may reach specific structures, such as the renal nerves, that are proximate to the lumens in which the catheters travel. Accordingly, catheter-based systems can deliver energy from within the lumens to inactivate the renal nerves in the vessel walls.

The ultrasound transducer can be mounted at a distal end of catheter, and the unfocused ultrasound energy can heat tissue adjacent to a body lumen within which the catheter (and the ultrasound transducer) is disposed. The system may also include a balloon mounted at the distal end of the catheter. The balloon is used to circulate cooling fluid both prior to, during, and after activation of the ultrasound transducer to cool the ultrasound transducer and help prevent thermal damage to the interior surface of the vessel wall while the nerves are being heated and damaged at depth.

### SUMMARY

The present disclosure is defined in the independent claims. Further embodiments of the present disclosure are defined in the dependent claims.

A fluid control device for an ultrasound-based treatment system is provided herein. The fluid control device includes at least one motor having a motor shaft extending from an axis of the motor. The at least one camshaft operatively coupled to the motor shaft in rotational communication with the motor shaft. The at least one actuator in communication with the at least one camshaft, such that when the motor shaft is operating, the motor shaft causes the at least one camshaft to rotate, thereby moving the at least one actuator between a first position and a second position through a fluid channel of a cartridge manifold.

A fluid control device for an ultrasound treatment system is provided herein. The fluid control device includes at least one rocker arm. The at least one actuator in communication with a first end of the at least one rocker arm. The at least one camshaft in communication with a second end of the rocker arm. The motor shaft extending from at least one motor, the motor shaft in rotational communication with the at least one camshaft such that when the motor shaft is operating, the motor shaft causes the at least one rocker arm to actuate thereby moving the at least one actuator between a first position and a second position.

A fluid control device for a balloon catheter is provided herein. The fluid control device for a balloon catheter includes at least one motor having a motor shaft extending from an axis of the motor. The at least one camshaft operatively coupled to the motor shaft in rotational communication with the motor shaft. The at least one actuator configured to move in communication with the at least one camshaft and control a flow of a fluid by pinching a flow path of the fluid to or from the balloon catheter.

The above summary does not include an exhaustive list of all aspects of the present disclosure. It is contemplated that the present disclosure includes all systems and methods that can be practiced from all suitable combinations of the various aspects summarized above, as well as those disclosed in the Detailed Description below and particularly pointed out in the claims filed with the application. Such combinations have particular advantages not specifically recited in the above summary.

### BRIEF DESCRIPTION OF THE DRAWINGS

The various features of the present disclosure and the manner of attaining them will be described in greater detail with reference to the following description, claims, and drawings, wherein reference numerals are reused, where appropriate, to indicate a correspondence between the referenced items, and wherein:

FIG. 1 is a perspective view of a catheter of a treatment system, in accordance with an embodiment.

FIG. 2 is a front perspective view of a generator and a fluid transfer cartridge of a treatment system, in accordance with an embodiment.

FIG. 3 is an exploded view of a cartridge shell of a fluid transfer cartridge, in accordance with an embodiment.

FIGS. 4A-4B are perspective views of a cartridge manifold, in accordance with an embodiment.

FIG. 5 is an exploded view of a cartridge manifold, in accordance with an embodiment.

FIG. 6 is a front view of a fluid transfer plate of a cartridge manifold, in accordance with an embodiment.

FIG. 7 is a rear view of a fluid transfer plate of a cartridge manifold, in accordance with an embodiment.

FIG. 8 is a perspective view of a piston of a cartridge manifold, in accordance with an embodiment.

FIG. 9 is a perspective view of a piston of a cartridge manifold, in accordance with an embodiment.

FIG. 10 is a sectional view, taken about line A-A of FIG. 7, of a piston of a cartridge manifold in a non-engaged position, in accordance with an embodiment.

FIG. 11 is a sectional view, taken about line A-A of FIG. 7, of a piston of a cartridge manifold in a closed position, in accordance with an embodiment.

FIG. 12 is a block diagram of a controller of a treatment system, in accordance with an embodiment.

FIG. 13 illustrates a more direct approach of the rotary pinch valve system, in accordance with an embodiment.

FIG. 14 illustrates a rotary pinch valve system using a lever/rocker arm, in accordance with an embodiment.

FIG. 15 illustrates a rotary pinch valve system comprising a third actuator, in accordance with an embodiment.

FIG. 16 illustrates a rotary pinch valve system comprising a vent, in accordance with an embodiment.

FIG. 17 illustrates a rotary pinch valve system at each fluid port, in accordance with an embodiment.

### DETAILED DESCRIPTION

Embodiments describe a treatment system having a generator and a fluid transfer cartridge, and methods of using the treatment system. The treatment system may be an ultrasound-based tissue treatment system, used to delivery unfocused ultrasonic energy radially outwardly to treat tissue within a target anatomical region, such as the renal nerves within a renal artery. Alternatively, the tissue treatment system may be used in other applications, such as to treat sympathetic nerves of the hepatic plexus within a hepatic artery. Thus, reference to the system as being a renal denervation system, or being used in treating, e.g., neuromodulating, renal nerve tissue is not limiting.

In various embodiments, description is made with reference to the figures. However, certain embodiments may be practiced without one or more of these specific details, or in combination with other known methods and configurations. In the following description, numerous specific details are set forth, such as specific configurations, dimensions, and processes, in order to provide a thorough understanding of the embodiments. In other instances, well-known processes and manufacturing techniques have not been described in particular detail in order to not unnecessarily obscure the description. Reference throughout this specification to “one embodiment,” “an embodiment,” or the like, means that a particular feature, structure, configuration, or characteristic described is included in at least one embodiment. Thus, the appearance of the phrase “one embodiment,” “an embodiment,” or the like, in various places throughout this specification are not necessarily referring to the same embodiment. Furthermore, the particular features, structures, configurations, or characteristics may be combined in any suitable manner in one or more embodiments.

The use of relative terms throughout the description may denote a relative position or direction. For example, “above” may indicate a first direction relative to a component. Similarly, “below” may indicate a second direction relative to the component, opposite to the first direction. Such terms are provided to establish relative frames of reference, however, and are not intended to limit the use or orientation of treatment system components, e.g., a fluid transfer cartridge or a generator, to a specific configuration described in the various embodiments below. The words “tubing,” “channel,” and/or “conduit” can be used interchangeably.

Existing hypertension treatment systems include generators to generate and deliver energy, e.g., RF or ultrasound energy, to a catheter-based intraluminal device. The treatment systems may also include components that engage with the generators to facilitate treatment. For example, cartridges may mount on the generators to deliver inflation or cooling fluid to a balloon mounted on an end of a catheter. The generator and/or cartridge may be large and bulky, especially in combination. Furthermore, mechanical and electrical connections between the generator and cartridge can be unreliable due to imperfect mounting, tolerance stack ups, or movement that occurs between the components during operation. In the case of cartridges that deliver fluids, the fluid transfer may not be accurately monitored either visually or automatically due to a lack of lighting in the procedure room and/or unstable sensor connections between the generator and the cartridge. The system may also integrate long lengths of internal tubing 1406 that increases an overall form factor of the equipment.

A linear solenoid valve can be used in the generator; however, it is bulky and requires a lot of power to actuate, while producing limited power. As such, it is desired to have a rotary pinch valve in the generator that is smaller and more power efficient. It is also desired to increase the force generated by the pinch valve, to produce more uniform force, to create less heat buildup in the generator enclosure, and to have a longer actuation stroke.

In an aspect, a treatment system for performing a medical procedure, e.g., renal denervation, is provided. The treatment system includes a fluid transfer cartridge to deliver fluid to a catheter, and a generator to deliver energy to the catheter. The fluid transfer cartridge and the generator combine to form a control unit of the treatment system. The control unit is compact. More particularly, the fluid transfer cartridge fits within a cartridge receptacle of the generator to form a clean and compact profile of the control unit. Furthermore, the fluid transfer cartridge has syringe components that can be fully contained within a cartridge housing to reduce an overall form factor of the control unit. The control unit is mechanically stable. The fluid transfer cartridge can be fastened to the generator by a fastening mechanism that evenly distributes a retention force around the cartridge housing, and which has a quick release mechanism to make engagement and disengagement of the components fast and reliable. The control unit is electrically stable. Electrical connections between the fluid transfer cartridge and the generator may be via spring-loaded electrical contact pins, commonly referred to as pogo pins. The spring-loaded pins can maintain pressure at the electrical contact points between the components such that the connections are resilient against relative movement that can occur during operation. Furthermore, sensors used to detect movement of system components, such as a syringe piston of the fluid transfer cartridge, may include position sensors, such as magnetic switches or optical sensors, that are more stable and less susceptible to misalignment than, for example, mechanical switches. The control unit is user-friendly. The control unit can include one or more processors and various sensors that operate to determine a system readiness state, e.g., whether various electrical or component connections have been made, and to provide feedback to a user. By way of example, the system can detect whether the fluid transfer cartridge is mounted in the cartridge receptacle of the generator and activate lights within the fluid transfer cartridge to illuminate the syringes to provide feedback about that state to the user.

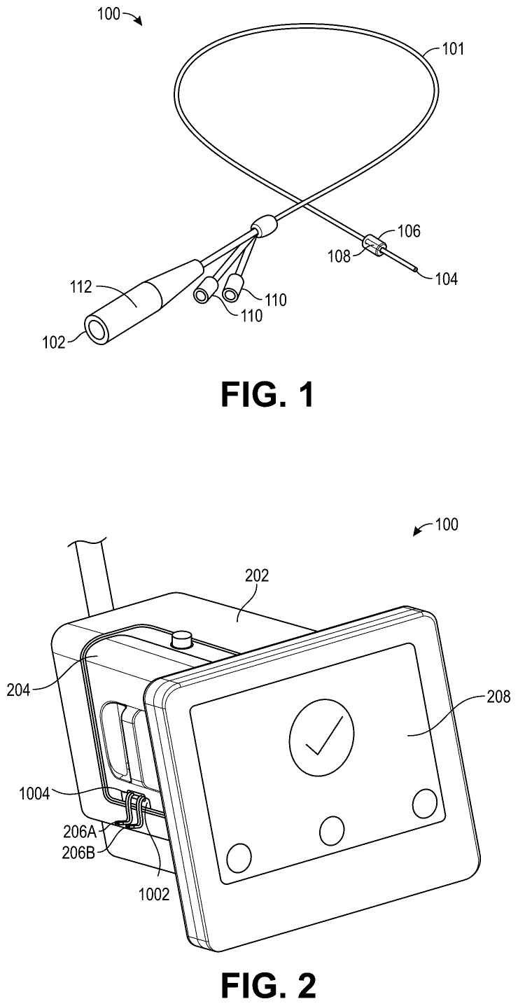

Referring to FIG. 1, a perspective view of a catheter-based intraluminal device of a treatment system is shown in accordance with an embodiment. The catheter-based intraluminal device of a treatment system 100 can include a catheter 101 having an elongated catheter body extending from a proximal catheter end 102 to a distal catheter end 104. An expandable member 106, such as a balloon, may be mounted on the catheter 101 at the distal catheter end 104. One or more energy transducers 108, such as ultrasound transducers, may be positioned within the expandable member 106. The expandable member 106 can be adapted to inflate within a target anatomy, e.g., a renal artery, and the energy transducer 108 can be adapted to deliver ablation energy, e.g., ultrasound energy, to the target anatomy during a medical procedure, e.g., a renal denervation procedure.

The catheter 101 can include one or more lumens, such as: fluid lumens to deliver an inflation/cooling fluid to the expandable member 106, electrical cable passageways containing electrical cables to deliver energy to the transducer 108, guidewire lumens for exchanging guidewires, etc. The lumen(s) may be connected to corresponding connectors at the proximal catheter end 102. For example, the fluid lumens may connect to one or more fluid ports 110, which receive inflation/cooling fluid from a fluid transfer cartridge of the treatment system 100, as described below. Similarly, the electrical cables can connect to an external connector 112, which receives energy from a generator of the treatment system 100, as described below.

Referring to FIG. 2, a front perspective view of a generator and a fluid transfer cartridge of a treatment system is shown in accordance with an embodiment. The treatment system 100 includes a control unit that connects to the catheter 101 to regulate the inflation of the balloon 106 with inflation/cooling fluid and to manage the delivery of ultrasound energy to the transducer 108. In an embodiment, the control unit includes a generator 202 to generate the ultrasound energy, and a fluid transfer cartridge 204 to transfer cooling fluid to and from the balloon 106 through one or more fluid conduits 206. For example, a fluid conduit 206A may transfer cooling fluid between a fluid reservoir, e.g., an intravascular fluid bag, and the fluid transfer cartridge 204. Similarly, a fluid conduit 206B can transfer cooling fluid between the fluid transfer cartridge 204 and the catheter 101. The control unit includes several other components, some of which are described below, to facilitate the energy and fluid transfer functions. Such components can include a display 208 to present procedural information to a user. Furthermore, the control unit can include one or more processors (not shown) configured to execute instructions stored in a memory device (not shown) to cause the treatment system 100 to perform various operations of the medical procedure, as described below. There is a conduit routing plate 1002 mounted in a conduit routing opening of a fluid transfer cartridge 204. The conduit routing plate comprises an upper edge 1004.

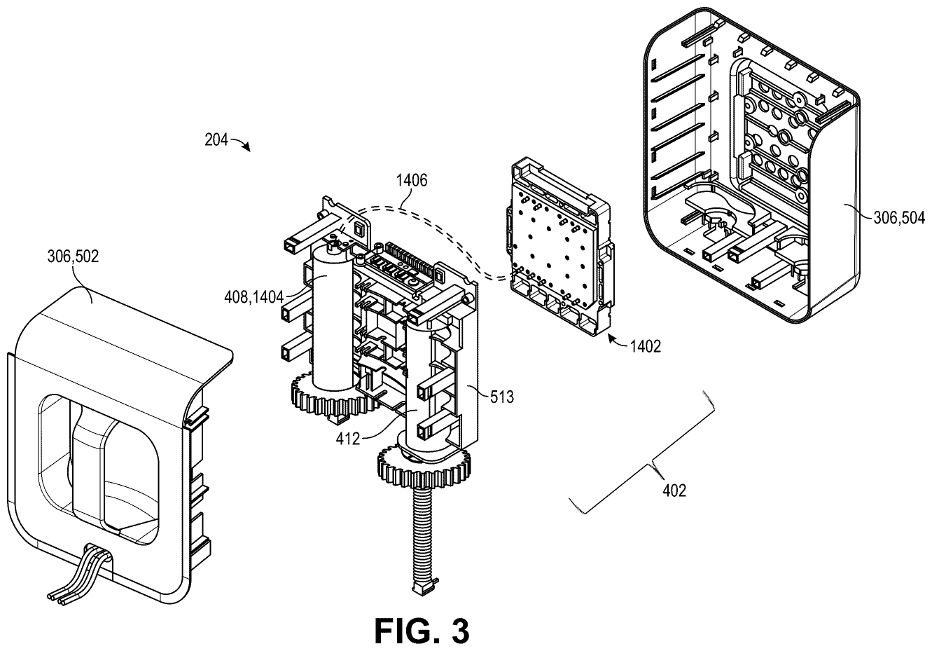

Referring to FIG. 3, an exploded view of a cartridge shell of a fluid transfer cartridge is shown in accordance with an embodiment. The cartridge shell 306 of the fluid transfer cartridge 204 can include the handle front plate 502 and the back plate 504. When combined, the handle front plate 502 and the back plate 504 can define the cartridge cavity 402 centrally located between the various walls and faces.

When snapped or otherwise fit together, the handle front plate 502 and the back plate 504 can contain, within the cartridge cavity 402, one or more components to provide fluid transfer functionality. For example, the fluid transfer cartridge 204 can include the syringe holder 513 to hold the syringe barrels 408, 412 within the cartridge cavity 402. The syringe holder 513 can stabilize the syringes during fluid delivery. The fluid transfer cartridge 204 can also include tubing 1406 to facilitate the movement of fluid from the syringes to the catheter 101.

The use of tubing 1406 to transfer fluid throughout the fluid transfer cartridge 204 may require long conduit lines and many glue joints to achieve the fluid pathway and interconnections that are needed for fluid transfer. For example, the exclusive use of tubing 1406 could require more than five feet of tubing 1406 and forty glue joints to create the fluid network. Such a fluid network, however, could occupy a substantial volume, could lead to leaks and/or flow inconsistency at the glued joints, and may be challenging to assemble during manufacturing. In an embodiment, a cartridge manifold 1402 may be used to replace much of the tubing 1406 length and joints, thereby providing a more compact, reliable, and easier to manufacture fluid network. Due to the reduced size and weight of the fluid network, the corresponding size and weight of the fluid transfer cartridge 204 may also be reduced, allowing more cartridges to be sterilized at once and more cartridges to be shipped per unit volume.

The cartridge manifold 1402 may replace some, but not all, of the fluid tubing 1406 within the fluid transfer cartridge 204. By way of example, the syringe barrel 408 may have a syringe cavity 1404, which is connected to fluid channels of the cartridge manifold 1402 by one or more conduits 1406. Other conduits 1406, e.g., between the cartridge manifold 1402 and the second syringe barrel 412, the balloon catheter 101, a fluid reservoir, etc., may also be routed through the cartridge cavity 402. Such conduits 1406 are not shown in FIG. 3 to avoid cluttering the illustration.

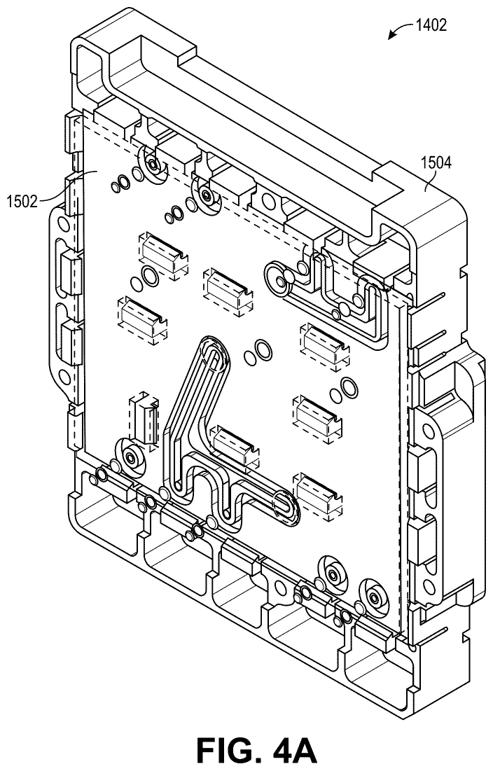

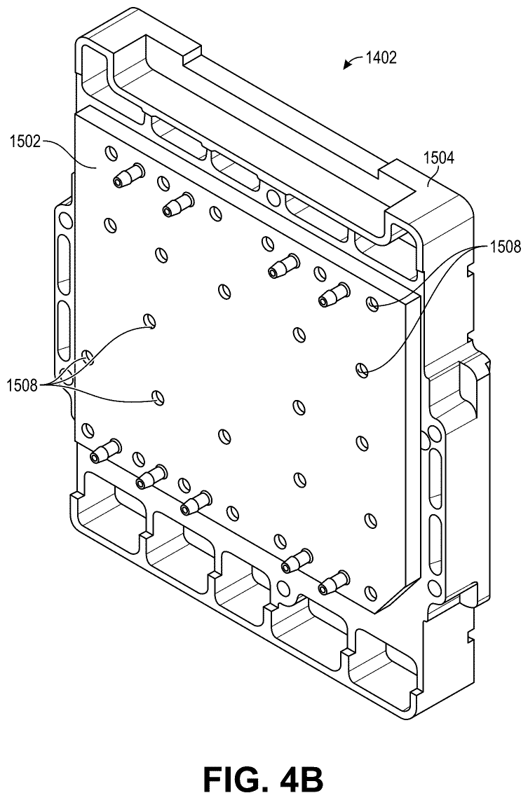

Referring to FIG. 4A, a perspective view of a cartridge manifold is shown in accordance with an embodiment. The cartridge manifold 1402 can include several plates assembled to each other. In an embodiment, the cartridge manifold 1402 includes a fore plate 1502 assembled to an aft plate 1504. The fore plate 1502 and the aft plate 1504 can be secured to each other. For example, the fore plate 1502 may be snap fit, e.g., secured by snap closures, to the aft plate 1504. As described below, the fore plate 1502 and the aft plate 1504 can secure an intermediate plate that has channels and ports to move cooling fluid throughout the cartridge manifold 1402, and to exchange the cooling fluid with external components such as the balloon catheter 101 and the fluid reservoir. The fore plate 1502 is rendered transparent to allow the intermediate plate to be viewed in FIG. 4A.

Referring to FIG. 4B, a perspective view of a cartridge manifold 1402 is shown in accordance with an embodiment. Alternatively, the fore plate 1502 and the aft plate 1504 may be fastened by screws or otherwise secured to each other. More particularly, several manifold fasteners 1508 can extend through through-holes in the aft plate 1504 and screw into threaded holes formed in the fore plate 1502. The fasteners 1508 can hold the plates together to sandwich an intermediate plate, as described below.

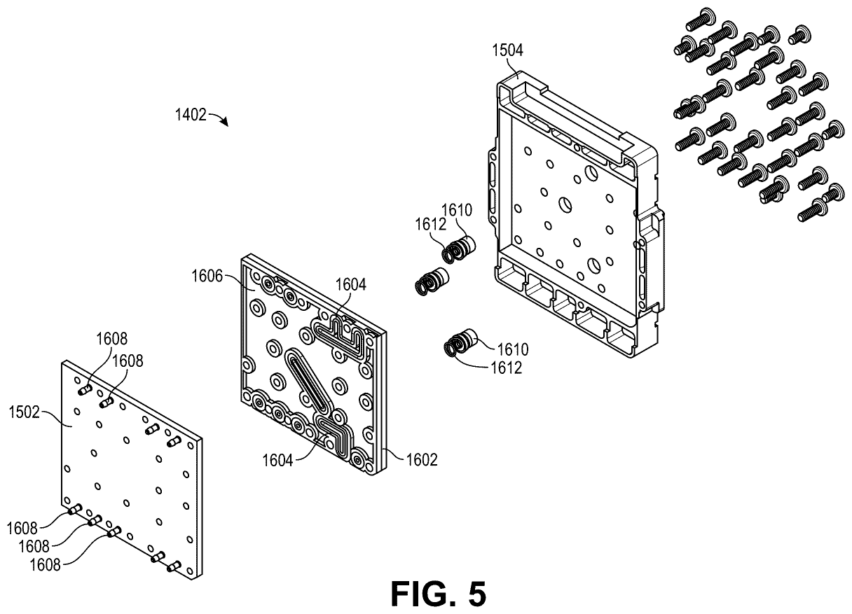

Referring to FIG. 5, an exploded view of a cartridge manifold is shown in accordance with an embodiment. The cartridge manifold 1402 in the cartridge cavity 402 can include a fluid transfer plate 1602 sandwiched between the fore plate 1502 and the aft plate 1504. The fluid transfer plate 1602 can include channels on front and rear surfaces that are connected through various ports in the plate. More particularly, one or more front fluid channel 1604 in a front plate surface 1606 can carry the cooling fluid, via channels on the rear surface, to one or more outlet ports 1608 for transfer to the external components.

In an embodiment, the outlet ports 1608 of the fore plate 1502 connect to external components. More particularly, the outlet ports 1608 can include fittings, e.g., barb fittings, that connect to fluid conduits 1406, and those conduits can extend to connect to external components, such as syringes, fluid reservoirs, the balloon catheter 101, or pressure sensors. Accordingly, the fore plate outlet ports 1608 can function as fluid interfaces to the external components. Through the outlet ports 1608, fluid can be transferred into and out of the cartridge manifold 1402. In an embodiment, the fore plate 1502 includes four outlet ports 1608 along an upper edge 1004 (FIG. 2) and five outlet ports 1608 along a lower edge 1004, although the number and locations of these outlet ports 1608 can be varied according to a layout of the external components and the fluid transfer cartridge 204.

Movement of the fluid through the tubing, channels, and ports of the cartridge manifold 1402 can be controlled by one or more pistons 1610. Each piston 1610 may be associated with, or include, a spring 1612. More particularly, the piston 1610 may be spring-loaded to bias the piston 1610 to a given position. For example, as described below, the spring 1612 may bias the piston 1610 to a non-engaged position and a solenoid can actuate the piston 1610 to move the piston 1610 to a closed position. In particular, the piston 1610 can be moved between positions that seal or unseal fluid ports in the fluid transfer plate 1602 to start or stop flow of the cooling fluid 603 through the fluid channels. A first position is a non-engaged position where the tubing 1406 is not pinched and there is fluid flow. A second position is a closed position where the tubing 1406 is pinched and there is no fluid flow. In certain embodiments, the tubing 1406 may be replaced by a channel or a tubing within a channel.

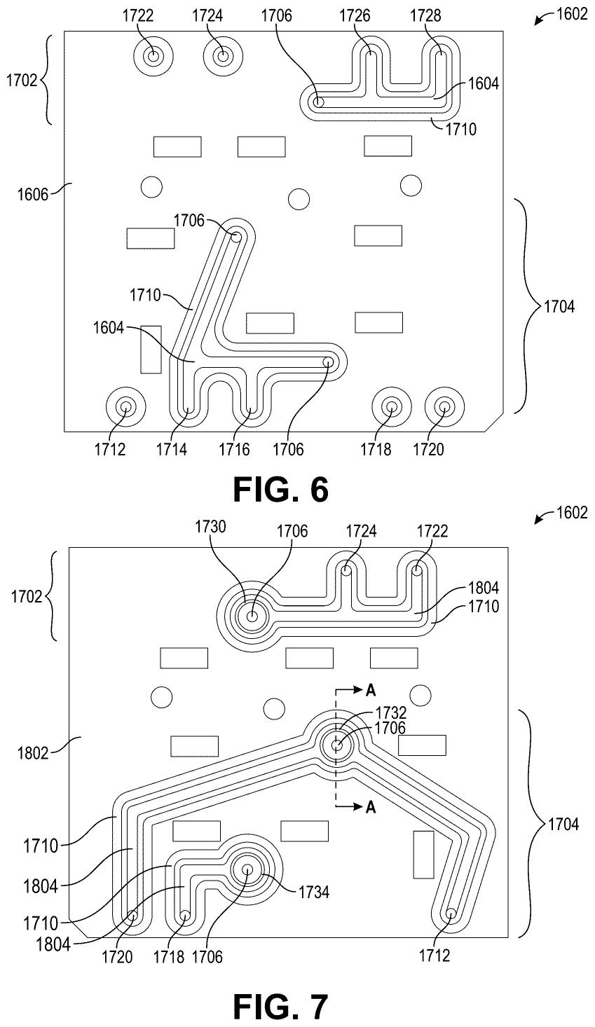

Referring to FIG. 6, a front view of a fluid transfer plate of a cartridge manifold is shown in accordance with an embodiment. The front plate surface 1606 of the fluid transfer plate 1602 can include several front fluid channels 1604. In an embodiment, the front fluid channels 1604 belong to respective fluid circuits. More particularly, some channels and ports may belong to an upper fluid circuit 1702, and other channels and ports may belong to a lower fluid circuit 1704. Each of the fluid circuits can include respective front fluid channels 1604 and one or more outlets. The fluid channels and outlets, as described below, can be interconnected with outlet ports 1608 of the fore plate 1502 to transfer fluid to and from external components. Furthermore, the fluid transfer plate 1602 can include one or more fluid port 1706. Each fluid port 1706 can extend through the fluid transfer plate 1602 from the front fluid channels 1604 in the front plate surface 1606 to rear fluid channels in a rear plate surface (FIG. 7). Accordingly, cooling fluid 603 can be moved from the channels in front of the fluid transfer plate 1602 to channels behind the fluid transfer plate 1602 through the fluid ports 1706.

In an embodiment, the fluid channels of the fluid transfer plate 1602 may be surrounded by respective channel seals 1710. The channel seals 1710 can be gaskets, e.g., O-rings, or strips of elastomeric material having circular, rectangular, cross-shaped, or other cross-sectional profiles, that are placed along an outer perimeter of the fluid channels. The seals can be fit into grooves, overmolded into the plate, or otherwise attached to the fluid transfer plate 1602. When the fluid transfer cartridge 204 is assembled, the channel seals 1710 can be sandwiched between the fluid transfer plate 1602 and an adjacent fore plate 1502 or aft plate 1504. The sandwiched seals can form a hermetic seal around the fluid channels to isolate the cooling fluid 603 within the channels.

The lower fluid circuit 1704 may be associated with the syringe barrel 408 that is used to deliver fluid to the balloon catheter 101. More particularly, cooling fluid 603 may be transferred from an external fluid reservoir, e.g., a fluid-filled bag, through the lower fluid circuit 1704 to the syringe barrel 408. The outlets of the front plate surface 1606, which connect to respective outlet ports 1608 of the fore plate 1502, can be labeled for ease of reference. For example, the lower fluid circuit 1704 can have an L1 outlet 1712, an L2 outlet 1714, an L3 outlet 1716, an L4 outlet 1718, and an L5 outlet 1720. Each of the L1-L5 outlets 1720 can connect to fittings on the fore plate 1502 that are in turn connected to conduits 1406. More particularly, the L1-L5 outlets can be in fluid communication with the outlet ports 1608 along the lower edge 1004 of the fore plate 1502. Those conduits/tubing 1406 may connect to external components such as the fluid reservoir, the syringe barrel 408, an inlet line of the balloon catheter 101, and/or one or more pressure sensors.

The upper fluid circuit 1702 may be associated with the second syringe barrel 412 that is used to draw fluid from the balloon catheter 101. More particularly, cooling fluid 603 may be transferred from the balloon catheter 101 through the upper fluid circuit 1702 to transfer the fluid to the external fluid reservoir. The outlets of the front plate surface 1606, which interconnect to respective outlet ports 1608 of the fore plate 1502, can be labeled for ease of reference. For example, the upper fluid circuit 1702 can have a U1 outlet 1722, a U2 outlet 1724, a U3 outlet 1726, and a U4 outlet 1728. Each of the U1-U4 outlets can connect to fittings on the fore plate 1502 that are in turn connected to conduits/tubing 1406. More particularly, the U1-U4 outlets can be in fluid communication with the outlet ports 1608 along the upper edge 1004 of the fore plate 1502. Those conduits/tubing 1406 may connect to external components such as an outlet line of the balloon catheter 101, the second syringe barrel 412, the fluid reservoir, and/or one or more pressure sensors.

It is apparent that some of the outlets are in fluid communication with each other through the fluid channels. For example, the U3 outlet 1726 and the U4 outlet 1728 are in fluid communication with each other through the front fluid channel 1604 of the upper fluid circuit 1702. Similarly, the L2 outlet 1714 and the L3 outlet 1716 are in fluid communication with each other through the front fluid channel 1604 of the lower fluid circuit 1704. As described below, the outlets that are isolated on the front side of the fluid transfer plate 1602, e.g., the U1, U2, L1, L4, and L5 outlets 1720, may also be in fluid communication with other outlets through fluid channels on the back side of the fluid transfer plate 1602. More particularly, each outlet and/or channel can include a respective fluid port 1706 extending through the fluid transfer plate 1602 to connect to corresponding channel(s) on the back side of the fluid transfer plate 1602.

Referring to FIG. 7, a rear view of a fluid transfer plate 1602 of a cartridge manifold is shown in accordance with an embodiment. The cartridge manifold 1402 includes a rear plate surface 1802 having one or more rear fluid channels 1804. Like the front fluid channels 1604, the rear fluid channels 1804 can be surrounded by channel seals 1710 to isolate fluid within the fluid channels. For example, the aft plate 1504 can be apposed to the rear plate surface 1802 such that the channel seals 1710 are sandwiched between the rear plate surface 1802 and the aft plate 1504. By extending around the rear fluid channels 1804, the channel seals can therefore define fluid pathways for transferring the cooling fluid 603.

The rear fluid channels 1804 belong to respective fluid circuits. More particularly, some channels and ports may belong to the upper fluid circuit 1702, and other channels and ports may belong to a lower fluid circuit 1704. The fluid channels and outlets on the rear plate surface 1802 can interconnect with the fluid channels and outlets on the front plate surface 1606 through the fluid ports 1706. In certain embodiments, instead of channels, tubing may be used, or tubing may be placed within the channels. More particularly, each fluid port 1706 can extend through the fluid transfer plate 1602 to interconnect the front fluid channels 1604 and ports to the rear fluid channels 1804 and ports. Similarly, given that the fluid channels and ports of the fluid transfer plate 1602 are connected to fittings on the fore plate 1502, which are in turn connected to the syringe barrel 408 through the conduit/tubing 1406, then the front fluid channel 1604, the rear fluid channel 1804, and the fluid ports 1706 are in fluid communication with the syringe cavity 1404. Accordingly, cooling fluid 603 can be moved between the syringe cavity 1404 and the channels in the fluid transfer plate 1602. Similarly, cooling fluid 603 can be moved between other external components and the channels in the fluid transfer plate 1602.

The outlets in the rear plate surface 1802 are labeled in FIG. 7 to show correspondence to the outlets in the front plate surface 1606 of FIG. 6. It is therefore apparent that the labeled outlets extend through the plate from the front plate surface 1606 to the rear plate surface 1802. More particularly, in the upper fluid circuit 1702, the U1 outlet 1722 and the U2 outlet 1724 are through-holes that extend through the plate. Similarly, in the lower fluid circuit 1704, the L1 outlet 1712, the L4 outlets 1718, and the L5 outlet 1720 are through-holes that extend through the plate. Accordingly, outlets that are isolated from each other on the front plate surface 1606 may be interconnected through the rear plate surface 1802. For example, the U1 outlet 1722 and the U2 outlet 1724 are physically isolated on the front plate surface 1606, however, those outlets are interconnected through the rear fluid channel 1804 on the rear plate surface 1802. Similarly, the L1 outlet 1712 and L5 outlet 1720 are physically isolated on the front plate surface 1606, however, those outlets are interconnected through the rear fluid channel 1804 on the rear plate surface 1802.

Whereas the fluid channels can interconnect outlets on one side of the plate that are isolated from each other on the other side of the plate, fluid ports 1706 can be used to reversibly interconnect fluid channels on one side of the plate with fluid channels on the other side of the plate. In an embodiment, each fluid port 1706 can be located within a corresponding valve seat on the rear plate surface 1802. The valve seats are labeled for ease of reference in the valve actuation logic described below. The upper fluid circuit 1702 can include a V1 valve seat 1730. The V1 valve seat 1730 can receive a corresponding piston 1610 to open and close the fluid port 1706 that interconnects the front fluid channel 1604 of the upper fluid circuit 1702 with the rear fluid channel 1804 of the upper fluid circuit 1702 at that location. Accordingly, the fluid port 1706 corresponding to the V1 valve seat 1730 can allow or stop fluid transfer between the front fluid channel 1604 and the rear fluid channel 1804 of the upper fluid circuit 1702. Thus, the fluid port 1706 corresponding to the V1 valve seat 1730 can cause the U1 and U2 outlets 1724 to be isolated from, or interconnected with, the U3 and U4 outlets 1728.

In an embodiment, the lower fluid circuit 1704 includes several valves seats. A V2 valve seat 1732 can receive a corresponding piston 1610 to open and close the fluid port 1706 that interconnects the front fluid channel 1604 of the lower fluid circuit 1704 with a first rear fluid channel 1804 of the lower fluid circuit 1704. The first rear fluid channel 1804 can interconnect the L1 outlet 1712 to the L5 outlet 1720. Accordingly, the fluid port 1706 corresponding to the V1 valve seat 1730 can allow or stop fluid transfer between the front fluid channel 1604 and the first rear fluid channel 1804 of the lower fluid circuit 1704. Thus, the fluid port 1706 corresponding to the V2 valve seat 1732 can cause the L2 and L3 outlets 1716 to be isolated from, or interconnected with, the L1 and L5 outlets 1720.

In an embodiment, a V3 valve seat 1734 can receive a corresponding piston 1610 to open and close the fluid port 1706 that interconnects the front fluid channel 1604 of the lower fluid circuit 1704 with a second rear fluid channel 1804 of the lower fluid circuit 1704. The second rear fluid channel 1804 can interconnect the fluid port 1706 at the V3 valve seat 1734 to the L4 outlets 1718. Accordingly, the fluid port 1706 corresponding to the V3 valve seat 1734 can allow or stop fluid transfer between the front fluid channel 1604 and the second rear fluid channel 1804 of the lower fluid circuit 1704. Thus, the fluid port 1706 corresponding to the V3 valve seat 1734 can cause the L2 and L3 outlets 1716 to be isolated from, or interconnected with, the L4 outlets 1718. It will also be appreciated, by examination of the illustrated fluid network, that actuating the pistons 1610 to simultaneously open the fluid ports 1706 at the V2 valve seat 1732 and the V3 valve seat 1734 would therefore place all the outlets of the lower fluid circuit 1704 in fluid communication with each other through the front fluid channel 1604, the first rear fluid channel 1804, and the second rear fluid channel 1804.

As described above, the fluid network formed by the various channels and ports of the fluid transfer plate 1602 can be used to interconnect various components external to the cartridge manifold 1402. An embodiment of external component connections is now described. Beginning with the upper fluid circuit 1702, the U1 outlet 1722 can connect to the second syringe barrel 412. Accordingly, transferring fluid through the U1 outlet 1722 can transfer fluid to or from the second syringe barrel 412. The U2 outlet 1724 can connect to the fluid reservoir. Accordingly, transferring fluid through the U2 outlet 1724 can transfer fluid to or from the fluid reservoir. The U3 outlet 1726 can connect to a pressure sensor. Accordingly, the U3 outlet 1726 can allow the fluid pressure in the front fluid channel 1604 (or the rear fluid channel 1804 of the upper fluid circuit 1702 when the corresponding valve is opened) to be sensed. The U4 outlet 1728 can connect to an outlet line of the balloon catheter 101. Accordingly, transferring fluid though the U4 outlet 1728 can transfer fluid to or from the outlet line of the balloon catheter 101.

With respect to the lower fluid circuit 1704, the L1 outlet 1712 can connect to an inlet line of the balloon catheter 101. Accordingly, transferring fluid through the L1 outlet 1712 can transfer fluid to or from the inlet line of the balloon catheter 101. The L2 outlet 1714 can connect to the syringe barrel 408. Accordingly, transferring fluid through the L2 outlet 1714 can transfer fluid to or from the syringe barrel 408. The L3 outlet 1716 can connect to a pressure sensor. Accordingly, the L3 outlet 1716 can allow the fluid pressure in the front fluid channel 1604 (or one or both rear fluid channels 1804 of the lower fluid circuit 1704 when the corresponding valves are opened) to be sensed. The L4 outlets 1718 can connect to the fluid reservoir. Accordingly, transferring fluid though the L4 outlets 1718 can transfer fluid to or from the fluid reservoir. The L5 outlet 1720 can connect to a pressure sensor. Accordingly, the L5 outlet 1720 can allow the fluid pressure in the first rear fluid channel 1804 of the lower fluid circuit 1704 (or one or both front fluid channel 1604 or the second rear fluid channel 1804 of the lower fluid circuit 1704 when the corresponding valves are opened) to be sensed.

Having described the fluid network and, in an embodiment, the external components connected to the fluid network, it is now possible to describe a method of circulating cooling fluid 603 from the fluid reservoir to the balloon catheter 101 and then back to the fluid reservoir. In a first operation, the fluid port 1706 at the V2 valve seat 1732 may be closed and the fluid port 1706 at the V3 valve seat 1734 may be opened. This closing/opening action can be produced by actuation of the pistons 1610, as described below. Alternatively, other valve designs may be integrated with the fluid transfer plate 1602 to open and close the respective fluid ports 1706.

At a first operation, with the V3 valve open, the L2, L3, and L4 outlets may be in fluid communication with each other, and the L1 and L5 outlets may be isolated from the other outlets in the lower fluid circuit 1704. Accordingly, the syringe piston 702 of the syringe barrel 408 may be retracted to draw fluid into the syringe cavity 1404 from the fluid reservoir. More particularly, the cooling fluid 603 can pass from the fluid reservoir into the L4 outlet 1718, through the fluid port 1706 at the V3 valve seat 1734, into the front fluid channel 1604, and out of the L2 outlet 1714 into conduit/tubing 1406 connected to the syringe barrel 408. At this stage, the pressure sensor connected to the L3 outlet 1716 can sense pressure of the transferred cooling fluid 603, e.g., in the syringe cavity 1404.

At a second operation, the V3 valve is closed and the V2 valve is opened. At this stage, the L1, L2, L3, and L5 outlets can be in fluid communication with each other, and the L4 outlet 1718 may be isolated from the other outlets in the lower fluid circuit 1704. Accordingly, the syringe piston 702 of the syringe barrel 408 can be advanced to push fluid out of the syringe cavity 1404 into the inlet line of the balloon catheter 101. More particularly, the cooling fluid 603 can pass from the syringe cavity 1404 into the L2 outlet 1714, through the fluid port 1706 at the V2 valve seat 1732, and out of the L1 outlet 1712 into the inlet line of the balloon catheter 101. At this stage, the pressure sensor connected to the L5 outlet 1720 can sense pressure of the transferred cooling fluid 603, e.g., in the balloon catheter 101.

At a third operation, with the V1 valve open, the U1, U2, U3, and U4 outlets may be in fluid communication with each other. Accordingly, the syringe piston 702 of the second syringe barrel 412 may be retracted to draw fluid into the syringe cavity 1404 from the outlet line of the balloon catheter 101. More particularly, the cooling fluid 603 can pass from the outlet line of the balloon catheter 101 into the U4 outlet 1728, through the front fluid channel 1604 and the fluid port 1706 at the V1 valve seat 1730, into the rear fluid channel 1804, and out of the U1 outlet 1722 into conduit 1406 connected to the second syringe barrel 412. The conduit 1406 connecting the U2 outlet 1724 to the fluid reservoir may have a one-way check valve that prevents backflow, and thus, no suction may be applied to the fluid reservoir at the U2 outlet 1724. At this stage, the pressure sensor connected to the U3 outlet 1726 can sense pressure of the transferred cooling fluid 603, e.g., in the syringe cavity 1404.

At a fourth operation, the V1 valve is closed. At this stage, the U1 and U2 outlets 1724 can be in fluid communication with each other, and the U3 and U4 outlets 1728 may be isolated from the other outlets in the upper fluid circuit 1702. Accordingly, the syringe piston 702 of the second syringe barrel 412 can be advanced to push fluid out of the syringe cavity 1404 into the fluid reservoir. More particularly, the cooling fluid 603 can pass from the syringe cavity 1404 into the U1 outlet 1722, through the rear fluid channel 1804 of the upper fluid circuit 1702, and out of the U2 outlet 1724 through the conduit/tubing 1406 (and the check valve) to fill the fluid reservoir.

The operations described above can be performed in series and/or in parallel to circulate cooling fluid 603 through the balloon catheter 101. For example, adding fluid to the balloon at the second operation can be performed simultaneously with removing fluid from the balloon at the third operation to balance positive and negative pressures in the balloon such that the balloon diameter remains constant while maintaining a temperature of the cooling fluid 603 within the balloon. Control of the operations can be provided in part based on pressure data fed back to one or more processors by the pressure sensors connected to the cartridge manifold 1402.

Referring to FIG. 8, a perspective view of a piston of a cartridge manifold is shown in accordance with an embodiment. The valves used to open and close the fluid ports 1706 can include the pistons 1610. More particularly, the pistons 1610 can interact with the fluid transfer plate 1602 to seal and unseal the fluid ports 1706. In an embodiment, the piston 1610 include an end seal 1902. As described below, the piston 1610 can be placed in a non-engaged position in which the end seal 1902 unseals (does not occlude) a corresponding fluid port 1706 to allow cooling fluid 603 to pass through the fluid port 1706. The piston 1610 can be moved from the non-engaged position to a closed position in which the end seal 1902 seals (occludes) the corresponding fluid port 1706 to stop the cooling fluid 603 from passing through the fluid port 1706. Accordingly, the piston 1610 acts as a valve by covering or uncovering the fluid port 1706 to control fluid flow therethrough.

In an embodiment, the end seal 1902 has a circular distal surface. The distal surface can be flat. The seal can include an elastomeric, cylindrical plug that is lodged into a body of the piston 1610. A face of the plug can extend distally from the body to seal against an opposing surface. For example, the end seal 1902 can press against the rear plate surface 1802 of the fluid transfer plate 1602. More particularly, the face of the end seal 1902 can seal against the rear plate surface 1802 at a corresponding valve seat surrounding the corresponding fluid port 1706 to close the valve. Accordingly, the end seal 1902 can be sized to be larger than the fluid port 1706. For example, a diameter of the face of the end seal 1902 may be larger than, e.g., twice as large as, a diameter of the fluid port 1706.

As described above, the piston 1610 can be spring-loaded. The piston 1610 may include a spring groove 1904. The spring groove 1904 can include an annular groove sized and shaped to receive a proximal end of the spring 1612. The spring 1612 can be a helical compression spring 1612. A distal end of the spring 1612 can be similarly engaged to a corresponding spring groove 1904 at the valve seat. The spring grooves 1904 can stabilize the spring 1612 and allow the spring 1612 to act against both the rear plate surface 1802 and the piston 1610. Accordingly, the spring 1612 can bias the piston 1610 outward to maintain the piston 1610 in a normally non-engaged position in which the end seal 1902 is offset from the rear plate surface 1802 to allow fluid flow through the fluid port 1706.

The piston 1610 can include a side seal 1906 to seal against one of the manifold plates. For example, the side seal 1906 can seal against the aft plate 1504. Accordingly, the piston 1610 can include the end seal 1902 to press against the rear plate surface 1802 of the fluid transfer plate 1602, and a side seal 1906 to seal against the aft plate 1504. In an embodiment, the side seal 1906 can include an O-ring that fits within a groove of the piston 1610 body. Thus, the end seal 1902 may have an annular distal surface. The O-ring can extend laterally beyond a cylindrical wall of the body, and thus, when the piston body is inserted into a receiving hole of the aft plate 1504, the side seal 1906 can press against and seal to the aft plate 1504. The side seal 1906 can maintain the seal while sliding against the aft plate 1504, and thus, the piston 1610 may be moved axially within the aft plate 1504. Accordingly, the piston 1610 can be advanced to occlude the corresponding fluid port 1706 or retracted to open the corresponding fluid port 1706.

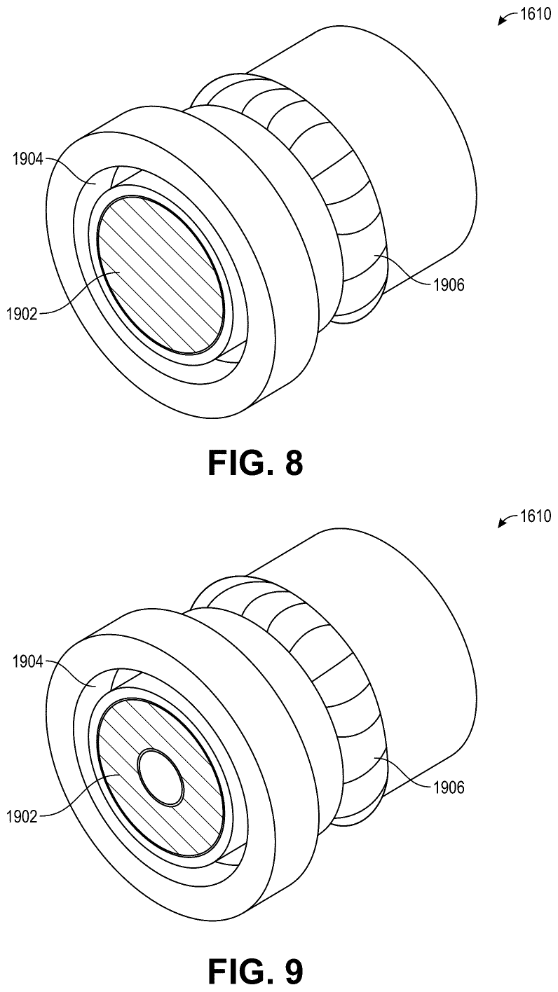

Referring to FIG. 9, a perspective view of a piston of a cartridge manifold is shown in accordance with an embodiment. The end seal 1902 may include an O-ring. The O-ring may be set within a groove in an end of the piston 1610. For example, the groove can be machined and the O-ring may be pressed into the groove. Alternatively, to create a more secure hold of the O-ring, the piston 1610 body may be overmolded around the O-ring. Accordingly, the end seal 1902 may be tightly secured within the piston 1610 body. In either case, the end seal 1902 can extend distally from the piston 1610 body such that the seal can press against the rear plate surface 1802 when the piston 1610 is moved to the closed position. An outer diameter of the annular end seal 1902 can be sized to be larger than the fluid port 1706. For example, the outer diameter of an O-ring end seal 1902 may be larger than, e.g., twice as large as, a diameter of the fluid port 1706.

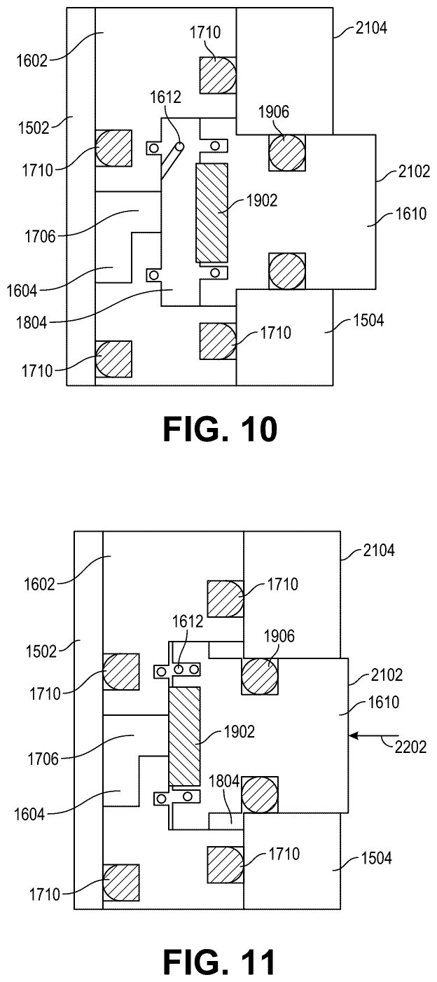

Referring to FIG. 10, a sectional view, taken about line A-A of FIG. 7, of a piston of a cartridge manifold 412 in a non-engaged position is shown in accordance with an embodiment. The piston 1610 can be a free floating piston 1610 having a side seal 1906 to radially seal against the aft plate 1504, as described above. Furthermore, the end seal 1902 can face the fluid port 1706 in the fluid transfer plate 1602. In the non-engaged position, however, the spring 1612 can maintain the end seal 1902 spaced apart from the fluid port 1706. Furthermore, the fluid pressure within the fluid channels in front of the end face 604 can press against the end face 604, biasing the piston 1610 to the non-engaged position. Accordingly, cooling fluid 603 may flow through the front fluid channel 1604 and the fluid port 1706 into the rear fluid channel 1804.

Referring to FIG. 11, a sectional view, taken about line A-A of FIG. 7, of a piston of a cartridge manifold in a closed position is shown in accordance with an embodiment. A solenoid 2202 (force vector shown, but solenoid 2202 omitted) can be actuated to press the piston 1610 forward. The solenoid 2202 force can overcome the spring 1612 and fluid pressure acting against the piston 1610 in an opposite direction to cause the piston 1610 to move to the closed position. In the closed position, the end seal 1902 obstructs the path of fluid flow through the fluid port 1706. More particularly, the cooling fluid 603 is stopped from flowing through the fluid port 1706 to or from the front fluid channel 1604.

Notably, the solenoid 2202 can close the valve using a force of less than 10 lbf, e.g., 5 lbf or less. Such closing force compares favorably relative to alternative valve designs, such as pinch valves that squeeze conduit/tubing 1406. However, the force that the solenoid 2202 produces is limited to how much it can affect the solenoid rod. Additionally, the rotary pinch valve comprises a gear train and motor 800, which provides the rotary pinch valve more closing force than typical pinch valve mechanisms. As a result, the cartridge manifold 1402 can also be designed to withstand lower compression forces, allowing for less material to be used in the design and a more compact form factor to be achieved.

The valve can be reversibly moved from the closed position of FIG. 11 to the non-engaged position of FIG. 10 by de-energizing the solenoid 2202. When the solenoid 2202 is no longer energized, the compression spring 1612 can act on the piston 1610 to return the piston 1610 to the non-engaged position.

As illustrated in FIGS. 10-11, a rear surface 2102 of the piston 1610 can be rearward of a back surface 2104 of the aft plate 1504 in both the non-engaged position (FIG. 10) and the closed position (FIG. 11). By maintaining the rear surface 2102 proud of the back surface 2104 in both piston 1610 positions, contact between the solenoids 2202 and the pistons 1610 is facilitated. More particularly, a likelihood of the solenoids 2202 losing contact with the rear surface 2102 is reduced because the rear surface 2102 does not recess into the hole in the aft plate 1504, below the back surface 2104.

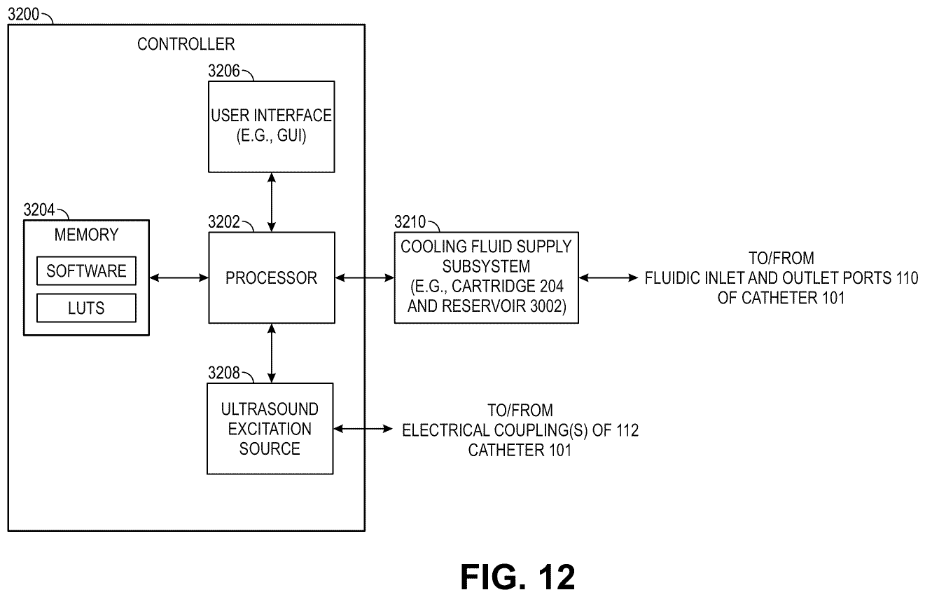

Referring to FIG. 12, a block diagram of a controller of a treatment system is shown in accordance with an embodiment. The block diagram represents an example implementation of the controller, which was introduced above. A controller 3200 is shown as including one or more processors 3202, a memory 3204, a user interface 3206, and an ultrasound excitation source 3208, but can include additional and/or alternative components. While not specifically shown, a processor 3202 can be located on a control board, or more generally, a printed circuit board (PCB) along with additional circuitry of the controller 3200. The processor 3202 can communicate with the memory 3204, which can include a non-transitory computer-readable medium storing instructions. The processor 3202 can execute the instructions to cause the treatment system 100 to perform the methods described herein. The user interface 3206 interacts with the processor 3202 to cause transmission of electrical signals at selected actuation frequencies to the ultrasound transducer 108 via wires of the connection cable and the cabling that extends through the catheter shaft 704. These wires electrically couple the controller 3200 to the transducer 108 so that the controller 3200 can send electrical signals to the transducer 108 and receive electrical signals from the transducer 108. The processor 3202 can control the ultrasound excitation source 3208 to control the amplitude and timing of the electrical signals to control the power level and duration of the ultrasound signals emitted by transducer 108. More generally, the controller 3200 can control one or more ultrasound treatment parameters that are used to perform sonication. In certain embodiments, the excitation source can also detect electrical signals generated by transducer 108 and communicate such signals to the processor 3202 and/or circuitry of a control board. While the ultrasound excitation source 3208 in FIG. 12 is shown as being part of the controller 3200, it is also possible that the ultrasound excitation source 3208 is external to the controller 3200 while still being controlled by the controller 3200, and more specifically, by the processor 3202 of the controller 3200.

The user interface 3206 can include a touch screen and/or buttons, switches, etc., to allow for an operator (user) to enter patient data, select treatment parameters, view records stored on a storage/retrieval unit (not shown), and/or otherwise communicate with the processor 3202. The user interface 3206 can include a voice-activated mechanism to enter patient data or may be able to communicate with additional equipment so that control of the controller 3200 is through a separate user interface 3206, such as a wired or wireless remote control. In some embodiments, the user interface 3206 is configured to receive operator-defined inputs, which can include, e.g., a duration of energy delivery, one or more other timing aspects of the energy delivery pulses (e.g., frequency, duty cycle, etc.), power, body lumen length, mode of operation, patient parameter, such as height and weight, and/or verification of artery diameter, or a combination thereof. Example modes of operation can include (but are not limited to): system initiation and set-up, catheter preparation, balloon inflation, verification of balloon apposition, pre-cooling, sonication, post-cooling, balloon deflation, and catheter removal, but are not limited thereto. In certain embodiments, the user interface 3206 provides a graphical user interface (GUI) that instructs a user how to properly operate the treatment system 100. The user interface 3206 can also be used to display treatment data for review and/or download, as well as to allow for software updates, and/or the like.

The controller 3200 can also control a cooling fluid supply subsystem 3210, which can include the fluid transfer cartridge 204 and fluid reservoir 3002, which were described above, but can include alternative types of fluid pumps, and/or the like. The cooling fluid supply subsystem 3210 is fluidically coupled to one or more fluid lumens (e.g., 110) within the catheter shaft which in turn are fluidically coupled to the balloon. The cooling fluid supply subsystem 3210 can be configured to circulate a cooling liquid through the catheter 101 to the transducer 108 in the balloon. The cooling fluid supply subsystem 3210 may include elements such as the fluid reservoir 3002 for holding the cooling fluid 603, pumps (e.g., syringes), a refrigerating coil (not shown), or the like for providing a supply of cooling fluid 603 to the interior space of the balloon at a controlled temperature, desirably at or below body temperature. The processor 3202 interfaces with the cooling fluid supply subsystem 3210 to control the flow of cooling fluid 603 into and out of the balloon. For example, the processor 3202 can control motor control devices linked to drive motors 1108 associated with pumps for controlling the speed of operation of pumps (e.g., syringes). Such motor control devices can be used, for example, where the pumps are positive displacement pumps, such as peristaltic pumps. Alternatively, or additionally, a control circuit may include structures such as controllable valves connected in the fluid circuit for varying resistance of the circuit to fluid flow (not shown). The processor 3202 can monitor pressure measurements obtained by the pressure sensors (e.g., P1, P2 and P3) to monitor and control the cooling fluid 603 through the catheter 101 and the balloon. The pressure sensors can also be used to determine if there is a blockage and/or a leak in the catheter 101. While the balloon is in an inflated state, the pressure sensors can be used to maintain a desired pressure in the balloon, e.g., at a pressure of between 10 psi and 30 psi, but not limited thereto. As will be described in additional detail below, the processor 3202 can use sensor measurements from one or more of the pressure sensors 2402 and/or other sensors to determine when the balloon is in apposition with a body lumen as well as to estimate an inner diameter of a body lumen to select an appropriate dose of ultrasound energy to be delivered to treat tissue surrounding the body lumen.

The controller 3200 can control operation of the generator 202 and fluid transfer cartridge 204 components to drive the inflation of the balloon prior to or during an interventional procedure. For example, the controller 3200 can control a priming process. The priming process can fill one or more of the syringes, the fluid manifold, the fluid conduit lines, and the balloon of the treatment system 100 with fluid and remove bubbles from the system. More particularly, the priming process can purge air from the fluidic system and prepare the treatment system 100 for delivery into the patient. The priming process may, as described below, include expelling fluid from a return syringe before filling an injection syringe to avoid sending air into the injection syringe. The controller 3200 may also control the inflation procedure, as described above, by driving the syringe piston 702 vertically to move the stopper 608 within the syringe and thus draw fluid into or expel fluid out of the syringe.

A position of the stopper 608 within the syringe can be determined by the controller 3200 based on several sensor inputs. The controller 3200 can receive feedback from the motor 1108 that drives the syringe piston 702 to determine stopper position. For example, the motor 1108 can provide data corresponding to a number of rotations of the gear 804, and the controller 3200 can determine, based on the rotations and known thread pitch information, a distance that the stopper 608 has been moved within the syringe. Furthermore, as described above, a magnetic or optical sensor can detect a location of the shaft end 706, e.g., a home position 1102. When the shaft end 706 is at the home position 1102, the stopper 608 can be at a known location within the syringe.

Although the motor 1108 feedback and home position sensor(s) can provide precise determination of the home position 1102, system slippage in the gear teeth or motor 1108 can lead to some inaccuracy as to whether the stopper 608 is accurately located at a same home position 1102 after each inflation/deflation cycle. More particularly, as the piston is driven upward and downward within the syringe over several cycles, the shaft end 706 may be driven based on motor 1108 rotations to a different home position 1102 in which the homing sensor is not triggered. When this happens, an error may be generated by the system. However, there may be only a marginally different amount of fluid remaining in the syringe when the error is triggered, as compared to when the stopper 608 was at the original home position 1102, which could create a nuisance requiring the user to re-home the system even when there is no practical impact to the system efficacy.

To avoid such nuisances, a homing process can be used that will dynamically adjust the home position when changes in the home position do not negatively affect system operation, and to generate an error when the changes may negatively affect system operation.

In an operation, the fluid transfer cartridge 204 is loaded into the generator 202. When operation begins, the shaft end 706 of the fluid transfer cartridge 204 will either be detected by the homing sensor, or not. If the shaft end 706 is not detected, then the controller 3200 can determine that the syringe must be homed prior to proceeding with fluidic priming and/or balloon inflation/deflation. If the shaft end 706 is detected, then the syringe can already be determined to be homed.

In the first case, when the shaft end 706 is not initially detected, the controller 3200 can drive the motor 1108 to raise the syringe pistons 702 until the shaft end 706 is detected by the position sensors. This is the initial home position. The controller 3200 can set the encoder volumes to zero at the initial home position. More particularly, the controller 3200 can determine a position value of the motor 1108 encoders and the position value can be set as the initial home position (corresponding to the home position of shaft end 706).

Switches include always on, always off, and intermittent (between always on and always off) positions. In an embodiment, the shaft end 706, upon reaching the initial home position, can be in either the always on or intermittent positions. If the initial home position is in the always on position, then lowering and raising the shaft end 706 to the initial home position should trigger a home position sensor. If the initial home position is in the intermittent position, then lowering and raising the shaft end 706 to the initial home position may or may not trigger a home position sensor.

It will be appreciated that, by monitoring shaft end position and motor encoders, a comparison of shaft end 706 location and remaining fluid volume can be performed. For example, at any location, the motor 1108 encoder information can be used to determine a stopper position and, thus, how much cooling fluid 603 remains in the syringe. In an embodiment, when the home position sensor is triggered, the controller 3200 can determine a remaining fluid volume in the syringe. When the remaining fluid volume is less than a predetermined volume, e.g., 3 to 5 mL, when the shaft end 706 is detected by the position sensor, even if the motor encoder is not at the same location as the initial home position, then the controller 3200 may set the motor position as a new home position. Alternatively, if the remaining volume is greater than the predetermined volume, e.g., greater than 5 mL, then the controller 3200 may generate an error to require the user to troubleshoot and re-home the system. In either case, the motor encoder can be used to determine cooling fluid volumes within the syringe at all states of the priming and/or inflation/deflation processes.

In the second case, when the shaft end 706 is initially detected, the controller 3200 can drive the motor 1108 to lower the syringe piston 702 to draw a predetermined volume of cooling fluid 603, e.g., 3 to 5 mL, into the syringe. The home position sensor can be monitored during the lowering process. If the home position sensor turns off during the lowering movement, then the motor encoder position can be set as a new home position by the controller 3200. The controller 3200 may proceed to control the system to perform the priming and/or inflation/deflation processes. Alternatively, if the home position sensor is still on after lowering the syringe to draw the predetermined volume of cooling fluid 603 into the syringe, then the controller 3200 can generate an error to require the user to troubleshoot and re-home the system. More particularly, the sensor remaining on after the lowering of the syringe likely indicates that the sensor has malfunctioned and the user may be notified accordingly. In either case, the motor encoder can be driven to perform the priming and/or inflation/deflation processes.

As mentioned above, a solenoid 2202 actuates the piston 1610 to pinch the tubing 1406 or channel or tubing within the channel. However, a lot of power is needed to produce enough force to pinch the tubing 1406. Additionally, as the piston 1610 actuates and moves out of the magnetic field, it starts to lose its force. For tubing 1406 that is rigid, almost 10 Watts of power per valve is needed to pinch the tubing 1406 to properly close it. Tubing 1406 experiencing higher pressure due to higher flow rate require more power to pinch. Further, the solenoid produces a lot of heat and requires a heat sink, whereas a heat sink is not used for the rotary pinch valve. As such, the rotary pinch valve system provides more power needed to pinch the tubing 1406 and the rod and coil mechanism is simple.

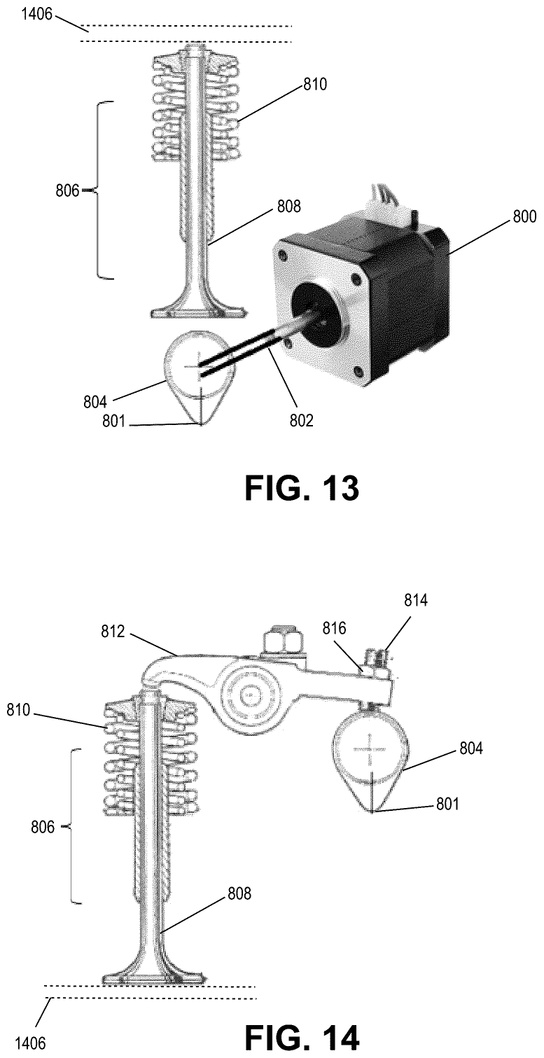

In the following figures, the tubing 1406 is drawn with dash lines to denote the environment. In the embodiments below, the actuator 806 will return to the passive position once the energy to the actuator 806 is removed. FIG. 13, illustrates a more direct approach of the rotary pinch valve system, in accordance with an embodiment. The system illustrated in FIG. 13 uses at least a motor actuator 806, camshaft 804, and a motor shaft 802. The camshaft 804 has an apex 801. The actuator 806 comprises a rod 808 and a spring 810. The spring 810, also known as a compression spring or a return spring, is used to ensure that the actuator 806 returns to its original position. The spring constant is designed to ensure that the mass of the actuator 806 and friction is overcome. In some embodiments, the elastic properties of the tubing 1406 or plastic materials can be used to create the same spring effect. The camshaft 804 is attached to the motor shaft 802, and when the motor 800 turns, the camshaft 804 pushes the rod 808 in one direction and the spring 810 pushes the rod 808 back. The rotary motion allows the camshaft 804 to move, and the movement of the camshaft 804 is used to either pinch the tubing 1406 by moving the actuator 806 to a second position/closed position or move the valve in the actuator of the manifold by actuating the piston 1610 to a second position/closed position.

In an embodiment, there is tubing 1406 that runs perpendicular to the actuator 806. On the opposite side of the actuator 806, there is a portion of the system that holds the tubing 1406 in place. As the camshaft 804 rotates, the camshaft 804 pushes the actuator 806 up, which pinches the tubing 1406 and stops fluid flow; also known as a second position/closed position.

The rotary approach allows gearing to be added to the motor shaft 802 and the gear will allow a larger force to be produced, (the bigger the gear ratio, the more forced produced to move the shaft) so more power is available to pinch the tubing 1406. For example, instead of utilizing a 10 Watt system, a 0.5 Watt system can be used to move the shaft effectively. Further, relatively unlimited power can be produced by changing the gear ratio of the mechanism. The gear ratio of the pitch circles of the mating gear defines the speed ratio and the mechanical advantage of the gear set. A gear train can be added to the motor 800, which would give it more force. To include a gear train, the motor 800 comprises gears that interface with gears in the camshaft 804. In an embodiment, the gear train has two gears. However, the quantity of gears may vary.

FIG. 14 illustrates another embodiment, where a lever arm/rocker arm is used in the rotary pinch valve system. The motor (FIG. 13) rotates the camshaft 804 and pushes the camshaft 804 up. The lever arm/rocker arm 812 can be used to develop mechanical advantage by pushing one side up while the other side moves down. The typical stroke length of the actuator 806 is dependent on the tubing 1406 size. In one embodiment, the tubing 1406 size can be 0.1 inches. Depending on the force needed, the distance from the center of the rocker arm 812 to the camshaft 804 will change. This also holds true with the distance from the center of the rocker arm 812 to the actuator 806. In other words, the longer the fulcrum effect, the higher the force. The speed of the motor 800 also depends on the force needed. For example, when a gear train is used, the gear train slows down the motor 800, but the force will increase. In this embodiment, the system does not necessarily need to be located as close to the piston arm because of the existence of the lever arm/rocker arm 812. Different types of camshafts 804 can be used, e.g., radial cam, cylindrical cam, wedge cam, conjugate cam, globoidal cam, or spherical cam. Different speeds of camshafts can be used. For example, a camshaft 804 can move up slower than it moves down or vice versa. The camshaft 804 may be attached to the rocker arm 812 using a lock nut 816 and an adjustment screw 814.

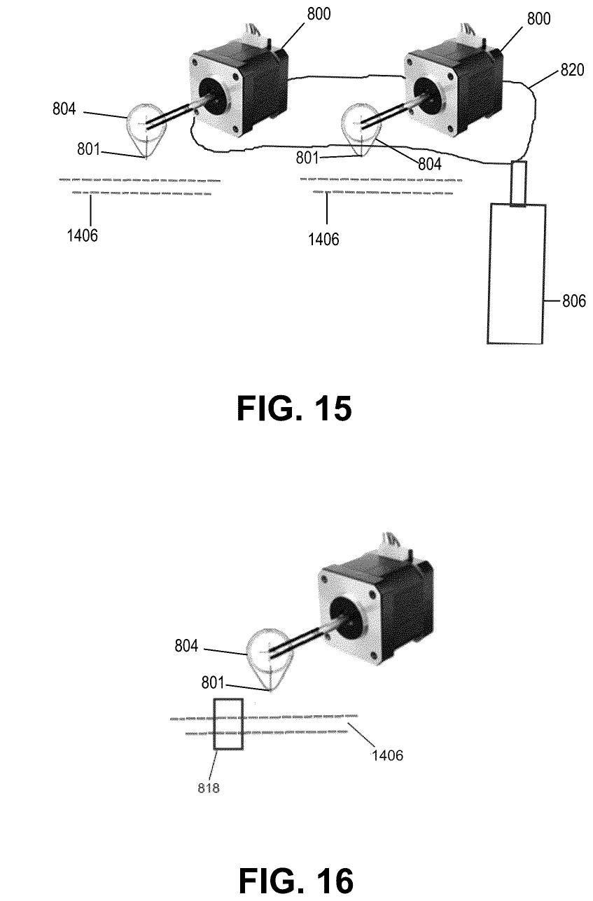

FIG. 15 illustrates a rotary pinch valve system comprising a third actuator, in accordance with an embodiment. Each motor 800 is located on a platform 820 and the third actuator 806 actuates the platform 820 away from the tubing 1406 when there is no power in the system. When there is no power, the tubing 1406 is in a first position (not pinched/non-engaged position) to deflate the catheter balloon. When there is power in the system, the third actuator 806 actuates the platform 820 towards the tubing 1406 and the tubing 1406 is in a second position (pinched/closed position). In this embodiment, the camshaft 804 pinches the tubing 1406 directly. As the camshaft 804 rotates, because of the irregular shape of the camshaft 804, the apex 801 moves towards the tubing 1406 due to rotary motion and pinches the tubing 1406. By having the camshaft 804 in the first position, this ensures that the pressure in the tubing 1406 is relieved when the power is not applied. The third actuator 806 will either move the camshaft 804 away from the tubing 1406, the tubing 1406 away from the camshaft 804, or move the camshaft 804 and the tubing 1406 away from each other. In an embodiment, the actuator 806 can be a solenoid that is either push or pull. There are two types of solenoids, ones that pulls the actuator into the field and others that push the actuator into the field when the solenoid is powered. FIG. 15 illustrates a solenoid that is a pull type.

FIG. 16 illustrates a rotary pinch valve system comprising a vent 818, in accordance with an embodiment. A vent 818 is a computer-controlled pressure relief vent that vents the tubing 1406 to atmospheric pressure when the power is removed. An example of a vent 818 can be a relief valve. The rotary pinch valve can be used to ensure that when the system is powered off there is no pressure in the tubing 1406. The relief valve will open and vent the system to atmosphere when the valve is not powered. In this embodiment, the camshaft 804 directly makes contact with the tubing 1406 or channel 1804 in the manifold.

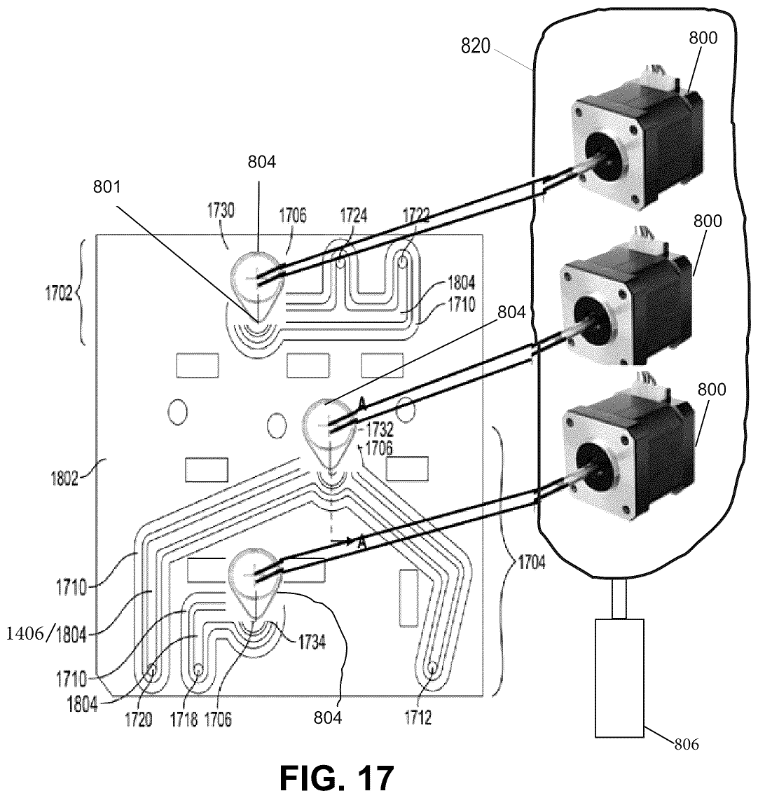

FIG. 17 illustrates a rotary pinch valve system at each fluid port 1706. In an embodiment, each motor 800 is located on a platform 820 and the actuator 806 actuates the platform 820 away from the tubing 1406/channel when there is no power in the system. When there is no power, the tubing 1406/channel is in a first position (not pinched/non-engaged position) to deflate the catheter balloon. When there is power in the system, the actuator 806 actuates the platform 820 towards the tubing 1406/channel and the tubing 1406/channel is in a second position (pinched/closed position). In this embodiment, the camshaft 804 pinches the tubing 1406/channel directly. As the camshaft 804 rotates, because of the irregular shape of the camshaft 804, the apex 801 moves towards the tubing 1406/channel due to rotary motion and pinches the tubing 1406/channel. By having the camshaft 804 in the first position, this ensures that the pressure in the tubing 1406/channel is relieved when the power is not applied. The actuator 806 will either move the camshaft 804 away from the tubing 1406/channel, the tubing 1406/channel away from the camshaft 804, or move the camshaft 804 and the tubing 1406/channel away from each other. In certain embodiments, there may be tubing 1406 located within a channel.

In another embodiment, the rear fluid channel 1804 comprises an actuator 806 and when the actuator 806 actuates, the camshaft 804 rotates and reaches its apex 801, thus pushing the actuator 806 towards the tubing 1406/channel and pinching the tubing 1406/channel (second position/closed position). As the camshaft 804 further rotates, the actuator 806 will be pulled out and the tubing 1406/channel is unpinched (first position/non-engaged position). In other words, the solenoid 2202 is replaced by the rotary pinch valve system. In certain embodiments, tubing 1406/channel, or tubing located within a channel may interact with the actuator 806.

In an embodiment, the at least one camshaft is a radial cam.

In an embodiment, the at least one camshaft is a cylindrical cam.

In an embodiment, the at least one camshaft is a wedge cam.

In an embodiment, the at least one camshaft is a conjugate cam.

In an embodiment, the at least one camshaft is a globoidal cam.

In an embodiment, the at least one camshaft is a spherical cam.

In the foregoing specification, the present disclosure has been described with reference to specific exemplary embodiments thereof. It will be evident that various modifications may be made thereto without departing from the broader spirit and scope of the present disclosure as set forth in the following claims. The specification and drawings are, accordingly, to be regarded in an illustrative sense rather than a restrictive sense.

What is claimed is:

1. A fluid control device for an ultrasound-based treatment system, comprising:
at least one motor having a motor shaft extending from an axis of the motor,
at least one camshaft operatively coupled to the motor shaft in rotational communication with the motor shaft, and
at least one actuator in communication with the at least one camshaft, such that when the motor shaft is operating, the motor shaft causes the at least one camshaft to rotate, thereby moving the at least one actuator between a first position and a second position through a fluid channel of a cartridge manifold.

2. The fluid control device of claim 1, further comprising one or more processors configured to control the at least one motor operably coupled to the at least one camshaft to move the at least one actuator between the first position and the second position.

3. The fluid control device of claim 1, wherein the at least one actuator is configured to move to the first position when power is removed.

4. The fluid control device of claim 1, further comprising a vent.

5. The fluid control device of claim 1, further comprising a gear train coupled to the motor shaft to produce more force at the second position.

6. The fluid control device of claim 1, wherein the at least one actuator is a rod and spring.

7. The fluid control device of claim 1, wherein the first position starts fluid flow.

8. The fluid control device of claim 1, wherein the second position stops fluid flow.

9. The fluid control device of claim 1, wherein the second position comprises an end seal to stop fluid flow through a fluid port.

10. The fluid control device of claim 9, wherein the fluid port is a front fluid channel.

11. The fluid control device of claim 1, further comprising a compression spring that acts on the at least one actuator to move the at least one actuator to an original position.

12. The fluid control device of claim 1, wherein the at least one camshaft is a radial cam.

13. A fluid control device for an ultrasound treatment system, comprising:
at least one rocker arm,
at least one actuator in communication with a first end of the at least one rocker arm,
at least one camshaft in communication with a second end of the rocker arm, and
a motor shaft extending from at least one motor, the motor shaft in rotational communication with the at least one camshaft such that when the motor shaft is operating, the motor shaft causes the at least one rocker arm to actuate thereby moving the at least one actuator between a first position and a second position.

14. The fluid control device of claim 13, further comprising one or more processors configured to control the at least one motor operably coupled to the camshaft to move the at least one actuator between the first position and the second position.

15. The fluid control device of claim 13, further comprising a gear train coupled to the motor shaft to produce more force at the second position.

16. The fluid control device of claim 13, wherein the at least one actuator is a rod and a spring.

17. The fluid control device of claim 13, wherein the first position starts fluid flow.

18. The fluid control device of claim 13, wherein the second position stops fluid flow.

19. The fluid control device of claim 13, wherein the second position comprises an end seal to stop fluid flow through a fluid port.

20. The fluid control device of claim 13, further comprising a compression spring that acts on the at least one actuator to move the at least one actuator to an original position.

21. A fluid control device for a balloon catheter, comprising:
at least one motor having a motor shaft extending from an axis of the motor,
at least one camshaft operatively coupled to the motor shaft in rotational communication with the motor shaft, and
at least one actuator configured to move in communication with the at least one camshaft and control a flow of a fluid by pinching a flow path of the fluid to or from the balloon catheter.

22. The fluid control device of claim 21, wherein the flow path is a flow tube.

23. The fluid control device of claim 21, wherein the flow path is a flow channel of a fluid manifold.
